(19) United States
(12) Patent Application Publication
(10) Pub. No.: US 2023/0271934 A1
(43) Pub. Date: Aug. 31, 2023

(54) PROCESS FOR PREPARATION OF SUBSTITUTED PYRAZOLES

(71) Applicants: Jie LI, Nanjing (CN); Avihai YACOVAN, Mazkeret Batya (IL); Bob CHEN, Minhang District, Shanghai (CN); Adama Makhteshim Ltd., Beer Sheva (IL)

(72) Inventors: Jie Li, Nanjing (CN); Avihai Yacovan, Mazkeret Batya (IL); Bob Chen, Shanghai (CN)

(73) Assignee: Adama Makhteshim Ltd., Beer Sheva (IL)

(21) Appl. No.: 18/020,317

(22) PCT Filed: Aug. 24, 2021
(86) PCT No.: PCT/IB2021/057766

§ 371 Date: Feb. 8, 2023

Related U.S. Application Data
(60) Provisional application No. 63/069,539, filed on Aug. 24, 2020.

(51) Int. Cl. C07D 401/04; C07D 231/16

(52) U.S. Cl. CPC C07D 401/04; C07D 231/16

(57) ABSTRACT

The present invention relates to the processes for the preparation of substituted pyrazole derivatives of formula (I), (II), (III), (IV), (IV-a) (V), (VII), which are useful as intermediates in anthranilamide insecticides production. In addition, the present invention is directed to a method for the preparation of anthranilamides of formula (VIII) using substituted pyrazole derivatives of formula (I), (III), (IV), (V), (VII). Furthermore, the present invention is directed to substituted halo-pyrazole compounds of formula (III), (IV) and (V), wherein X is halogen and R⁷ is hydrogen, C₁-C₄ alkyl.

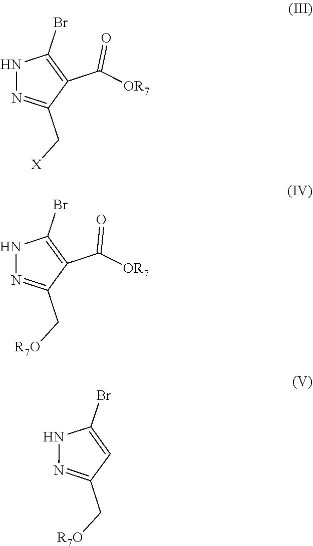

### TECHNICAL FIELD

The present invention concerns an improved process for preparation of substituted pyrazole derivatives and to novel halo-pyrazole derivatives which are useful for preparation of certain anthranilic amide compounds that are of interest as insecticides.

### SUMMARY OF INVENTION

The present invention accordingly relates to a process for preparation of compound of formula I,

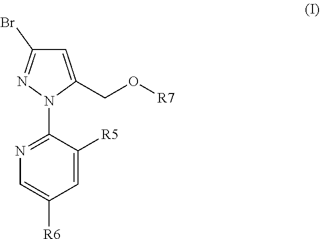

Wherein R⁵ is H, F, Cl or Br; and

R⁶ is H, F, Cl or Br; R⁷ is C₁-C₄ alkyl comprising:

a) reaction of compound of formula (II) with brominating agent, optionally in the presence of organic solvent

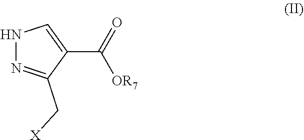

to prepare a compound of formula (III)

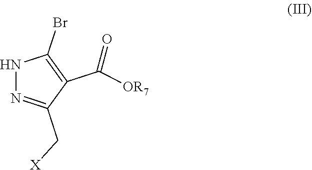

b) alkoxylation of compound of formula (III) in the presence of base to prepare a compound of formula (IV)

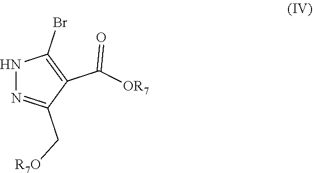

Or, alternatively, a) reaction of compound of formula (II) with alkoxylating agent in the presence of organic solvent

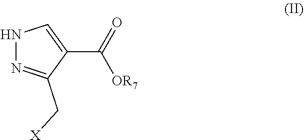

to prepare a compound of formula (IV-a),

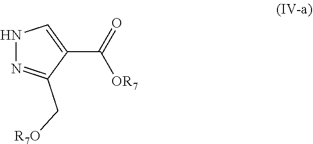

b) bromination of compound (IV-a) to prepare a compound of formula (IV),

c) decarboxylation of compound of formula (IV) to prepare a compound of formula (V):

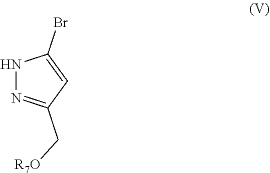

wherein X is halogen and R⁷ is hydrogen, C₁-C₄ alkyl; d) reaction of pyridine of formula (VI)

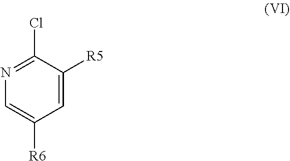

Wherein R⁵ is H, F, Cl or Br; and

R⁶ is H, F, Cl or Br; R⁷ is C₁-C₄ alkyl

with compound of formula (V) in the presence of base.

The brominating agent according to the above process is selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric acid (=DBI), bromodimethylsulfonium bromide, 5,5-dibromomeldrum's acid CAS RN: 66131-14-4, bis(2,4,6-trimethylpyridine)-bromonium hexafluorophosphate, bromine monochloride and the mixtures thereof.

The base according to the above process is selected from the group consisting of sodium methoxide, potassium methoxide, sodium ethoxide, potassium ethoxide, potassium tert-butoxide, lithium tert-butoxide, potassium carbonate, sodium bicarbonate, potassium bicarbonate, sodium carbonate, lithium carbonate, sodium hydroxide, lithium hydroxide, potassium hydroxide, sodium acetate, potassium acetate and the mixtures thereof.

In the aforementioned process the organic solvent is selected from the group consisting of polar or non-polar organic solvents such as C₁-C₆ alcohols, ketones, esters, aromatic solvents, heteroaromatic solvents, aliphatic solvents, amides, sulfones, sulfoxides, halogenated solvents, nitriles, carbonates, ureas and mixtures thereof. The suitable polar solvent can be, for example but not limited to, alcohol (preferably C₁-C₄ alcohol), acetone, acetonitrile, tetrahydrofuran, dimethyl sulfoxide, N, N-dimethylformamide, N, N-dimethylacetamide, N,N-dimethylethanolamine or a mixture thereof. In an embodiment, a suitable solvent consisting of N, N-dimethylacetamide, N, N-dimethylformamide, dimethyl sulfoxide, n-butanol, ethanol and the mixtures thereof.

In the preparation of compound (IV-a) according to the above process the alkoxylation step is performed in the presence of alkoxides selected from the group consisting of alkali metal oxides of C₁-C₄ alcohols, e.g., sodium methoxide, potassium methoxide, sodium ethoxide, potassium ethoxide, sodium tert-butoxide, and the mixtures thereof. Alternatively, the alkoxylation step can be carried out in the presence of C₁-C₄ alcohols and alkali metal carbonates, bicarbonates, hydroxides, and the mixtures thereof.

In addition, the present invention is directed to the process for preparation of compound (VII)

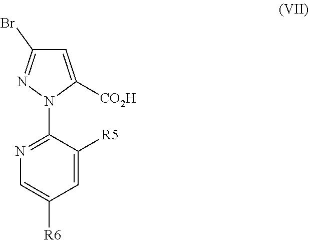

comprising reaction of oxidant with compound of formula (I) optionally in the presence of a catalyst, wherein the oxidant is selected from the group consisting of oxygen, air, ozone, hydrogen peroxide, benzoyl peroxide, tert-butyl peroxide, m-chloroperoxybenzoic acid, peroxyacetic acid, peroxybenzoic acid, magnesium monoperoxyphthalate, potassium peroxy monosulfate sodium permanganate, potassium permanganate and mixtures thereof.

The catalyst optionally used in the aforementioned oxidation reaction can be selected from the group consisting of N-hydroxysuccinimide, N-hydroxyphthalimide, N-hydroxybenzotriazole, tetraethylammonium hydrogensulfate, triethylbenzylammonium chloride, tetraphenylphosphonium bromide, PEGs, crown ethers, sodium nitrite, tert-butyl nitrite, cobalt(II) acetate, manganese(II) acetate, sodium nitrite, tert-butyl nitrite and the mixtures thereof.

Optionally, the process for preparation of compound (VII) is performed in the presence of organic solvent selected from the group consisting of C₁-C₆ alcohol, carboxylic acids and esters thereof, chlorinated hydrocarbons, sulfoxides, sulfones, amides, ethers, ketones, pyridine, and the mixtures thereof.

This invention also relates to compounds of formulae (III), (IV) and (V) and their use in improved processes of preparing of compounds of Formulae I, VII, VIII.

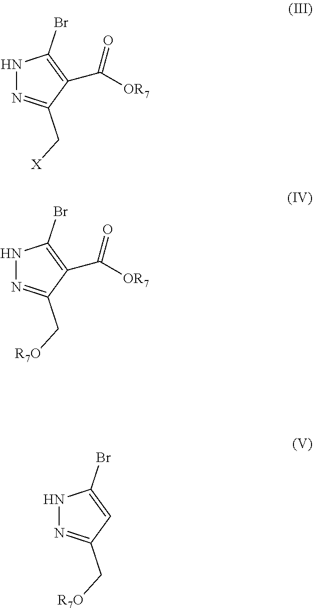

Wherein X and R⁷ are as defined above.

The present invention also pertains to a method of preparation of anthranilamide of formula (VIII)

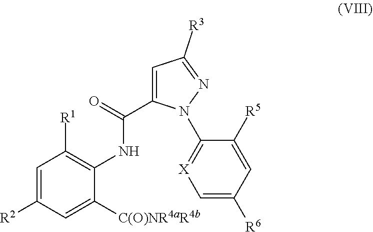

wherein

X is N; R¹ is CH₃, CI, Br or F;

R² is H, F, Cl, Br or CN;

R³ is Br;

R^{4a} is H, C₁-C₄ alkyl, cyclopropylmethyl or 1-cyclopropylethyl;

R^{4b} is H or CH₃; R⁵ is H, F, Cl or Br; and

R⁶ is H, F, CI or Br,

wherein the improvement comprising the compounds of formulae (I), (III) (IV), (V) prepared by the methods as indicated above.

In addition, the present invention is directed to a method of preparation of anthranilamide of formula (VIII) wherein X, R¹, R², R³, R^{4a}, R^{4b}, R⁵ and R⁶ are as indicated above, wherein the improvement comprising the compound of formula (VII) prepared from compound of formula (I) as indicated above.

### BACKGROUND

Certain anthranilamide compounds and methods of their preparation using different pyrazole precursors are known, for example from WO 2001/70671, WO 2003/015518, WO 2003/015519, WO 2004/067528, WO 2004/011447.

Among pyrazole precursors, of mention are substituted pyrazole carboxylic acids. Different methods of their preparation disclosed, however, all these methods include complicated multistep processes. For example, in WO 2003/015519, the preparation of said pyrazole carboxylic acid precursors of anthranilamides involves the reaction of substituted pyrazoles with a 2,3-dihalopyridine to produce 1-pyridylpyrazole and further metalation of 1-pyridylpyrazole with lithium diisopropylamide followed by quenching of the lithium salt with carbon dioxide.

In WO 2003/016283, the pyrazole carboxylic acid precursors of anthranilamides are prepared by oxidation of the corresponding substituted dihydro-1H-pyrazoles, which, in turn are prepared by a multistep process including complicated workup and low industrial applicability.

A process for making the substituted pyrazoles of formula (I) is known from WO 2008/126933. However, the process disclosed in WO 2008/126933 on Scheme 10 has drawbacks, for example, low yields and complicated workup; therefore, a need exists for more efficient industrially applicable processes for manufacturing of important intermediates of formula (I). Novel substituted pyrazoles of formula (III), (IV), (V) are not reported in the literature. Said substituted pyrazoles are useful chemical intermediates which are prepared from commercially available raw materials in high yields and good quality in an economically advantageous and easily handled way.

Based on the above, it would be highly desirable to provide an improved process for the production of the compound of formula (VIII) which is suitable for industrial use, highly efficient, low-cost, environmentally friendly, and provides a high yield and overcomes the drawbacks of the known processes.

### DESCRIPTION

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

The term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one skilled in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, use of the term “about” herein specifically includes ±10% from the indicated values in the range. In addition, the endpoints of all ranges directed to the same component or property herein are inclusive of the endpoints, are independently combinable, and include all intermediate points and ranges.

In the present invention, the term “alkyl”, used either alone or in compound words such as “alkylthio” or “haloalkyl” includes straight-chain or branched alkyl, such as methyl, ethyl, n-propyl, iso-propyl, or the different butyl, pentyl or hexyl isomers.

Certain compounds of this invention can exist as various stereoisomers including enantiomers, diastereomers, and geometric isomers. It is known in the art that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. Accordingly, the compounds of the invention may be present as a mixture of stereoisomers, individual stereoisomers, or as an optically active form.

The process for preparation of compound of formula I provided herein,

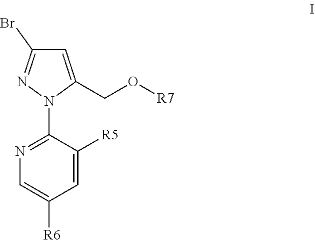

wherein R⁵ is H, F, Cl or Br; R⁶ is H, F, Cl or Br,

R⁷ is hydrogen, C₁-C₄ alkyl; comprises: a) reaction of compound of formula (II) with brominating agent, optionally in the presence of organic solvent

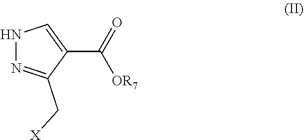

to prepare a compound of formula (III)

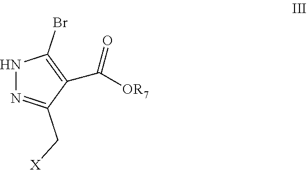

In step (a), a compound of Formula II is treated with a brominating agent, optionally in the presence of organic solvent. Polar and non-polar organic solvents can be used, wherein among polar solvents C₁-C₆ alcohols, acetonitrile, tetrahydrofuran, N,N-dimethylformamide, dimethyl sulfoxide and the like are suitable. Among non-polar solvents toluene, chlorobenzene, dichloromethane, dichloroethane, chloroform and the like are suitable. Two or more of the above-mentioned solvents may be used as a mixture, and the reaction may be performed in a single-phase system or a two-phase system. Preferred solvents are alcohols such as methanol, ethanol, tert-butanol and mixtures thereof. Additional suitable solvents are acetonitrile, ethanol and mixtures thereof. The reaction temperature is typically between 0° C. and the boiling point of the solvent, and the reaction time is typically from 2 to 20 hours.

The reaction mass is then neutralized with an inorganic base, such as sodium bicarbonate, sodium hydroxide and the like, or an organic base, such as sodium acetate. The desired product, a compound of Formula III, can be isolated by methods known to those skilled in the art, including crystallization, extraction and distillation.

The compound of Formula (II) is commercially available or can be prepared by known methods, recited for example, in DE3934924 and WO 2012/025469. As an example, the compound of formula (II) could be prepared similar to known method from ethyl (E)-4-chloro-2-((dimethylamino)methylene)-3-oxobutanoate by reaction with hydrazine according to Scheme I:

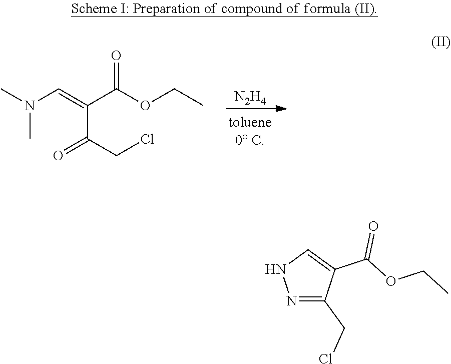

The starting ethyl (E)-4-chloro-2-((dimethylamino)methylene)-3-oxobutanoate could be prepared by known method as shown for example on Scheme II:

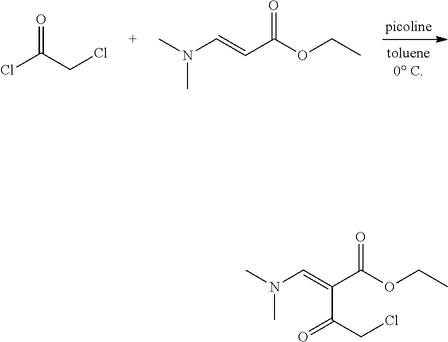

### Scheme II: Preparation of (E)-4-chloro-2-((dimethylamino)methylene)-3-oxobutanoate

In step b) according to the invention, the compound of formula (III) is reacted with alkoxylating agent to prepare a compound of formula (IV)

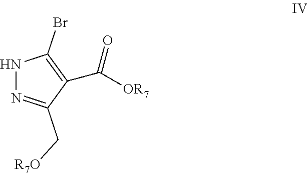

wherein R⁷ is as defined above.

In the preparation of compound (IV) according to the above process the alkoxylation step is performed in the presence of alkoxides selected from the group consisting of alkali metal oxides of C₁-C₄ alcohols, e.g., sodium methoxide, potassium methoxide, sodium ethoxide, potassium ethoxide, sodium tert-butoxide, and the mixtures thereof. Alternatively, the alkoxylation step can be carried out in the presence of C₁-C₄ alcohols and alkali metal carbonates, bicarbonates, hydroxides, and the mixtures thereof.

Greater than 1.0 equivalents of alkoxylating agent versus the compound of Formula III should be used, preferably between 1 and 10 equivalents. The reaction temperature is typically between −10° C. to 40° C. The resulting compound of Formula IV, can be isolated by methods known to those skilled in the art, including crystallization, extraction and distillation.

Alternatively, the compound of Formula IV is prepared by a) reaction of compound of formula (II) with base in the presence of polar organic solvent to prepare a compound of formula (IV-a), and further bromination of compound (IV-a) to prepare a compound of formula (IV):

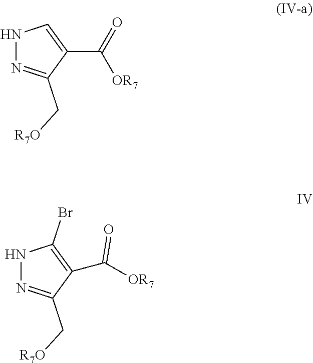

In the step c) according to the present invention, the compound of Formula (V) is prepared by decarboxylation of compound of formula (IV):

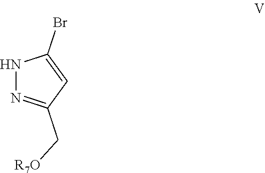

According to the present invention, the decarboxylation reaction is performed by heating the compound of Formula IV preferably to a temperature of 90° to 120° C., more preferably to a temperature of 100-105° C. with 30-60% vol of acid such as hydrochloric acid, hydrobromic acid, tetrafluoroboric acid, hexafluorophosphoric acid, trifluoroacetic acid, sulfuric acid, sulfonic acid, sulfinic acid, phosphoric acid, phosphonic acid and the mixtures thereof.

For the reaction, catalytic amounts of acid are generally sufficient. In general, the acid is used in an amount of from 0.1 to 1000 mole and especially in the amount of from 1.0 to 10.0 mole per mole of compound of formula (IV).

Typically, the decarboxylation reaction is employed in the presence of an organic solvent or solvent mixture. Suitable organic solvents are protic polar solvents, for example aliphatic alcohols having preferably from 1 to 4 carbon atoms, such as methanol, ethanol, n-propanol, isopropanol, n-butanol, isobutanol or tert-butanol, or carboxylic acids such as acetic acid, or aromatic polar solvents such as aromatic hydrocarbons such as benzene, toluene, xylenes, cumene, chlorobenzene, nitrobenzene or tert-butylbenzene, aprotic polar solvents, for example cyclic or acyclic ethers such as diethyl ether, diisopropyl ether, tert-butyl methyl ether (MTBE), tert-butyl ethyl ether, tetrahydrofuran (THF) or dioxane, cyclic or acyclic amides such as dimethylformamide, dimethylacetamide, N-methylpyrrolidone or tetramethylurea, or aliphatic nitriles such as acetonitrile or propionitrile, and mixtures thereof.

In the step d) according to the present invention, the pyridine of formula (VI) is reacted with the compound of formula (V) in the presence of base. The base could be selected from the group consisting of alkaline and earth alkaline hydroxides, hydrides, alkoxides and salts of sulfuric, sulfonic, sulfinic, phosphoric, phosphonic, formic, oxalic, carbonic, acetic, propionic, benzoic, and citric acid. More preferably, the suitable base can be alkali metal carbonate and/or alkali metal hydroxide.

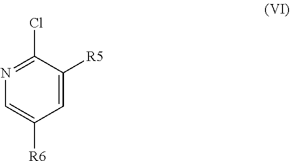

Wherein R⁵ is H, F, Cl or Br; and

R⁶ is H, F, Cl or Br.

The amount of the base employed is selected from a value in the range between 0.01, and 10.0 molar equivalents with respect to starting compound of formula (V).

Alternatively, in step a), the compound of Formula IV-a is produced by the alkoxylation of compound of formula (II) with base in the presence of organic solvent. In the preparation of compound (IV-a) according to the above process the alkoxylation step is performed in the presence of alkoxides selected from the group consisting of alkali metal oxides of C₁-C₄ alcohols, e.g., sodium methoxide, potassium methoxide, sodium ethoxide, potassium ethoxide, sodium tert-butoxide, and the mixtures thereof. Alternatively, the alkoxylation step can be carried out in the presence of C₁-C₄ alcohols and alkali metal carbonates, bicarbonates, hydroxides, and the mixtures thereof.

Examples of the organic solvent include ethers such as 1,4-dioxane, diethyl ether, tetrahydrofuran, methyl tert-butyl ether and the like, halogenated hydrocarbons such as dichloromethane, chloroform, carbon tetrachloride, 1,2-dichloroethane, chlorobenzene and the like, hydrocarbons such as toluene, benzene, xylene and the like, nitriles such as acetonitrile and the like, aprotic polar solvents such as N,N-dimethylformamide, N-methylpyrrolidone, 1,3-dimethyl-2-imidazolidinone, dimethyl sulfoxide and the like, alcohols such as methanol, ethanol, isopropyl alcohol and the like, and the mixtures thereof. In a further conversion, a bromination of compound (IV-a) gives compound of Formula (IV).

The brominating agent used in step a) is selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric Acid (=DBI), bromodimethylsulfonium bromide, 5,5-dbromomeldrum's acid CAS RN: 66131-14-4, Bis(2,4,6-trimethylpyridine)-bromonium hexafluorophosphate, bromine monochloride and the mixtures thereof.

In the practice of this invention, reaction temperatures are maintained in the range of from 0° C. to 100° C. and preferably in the range of 15 to 30° C. for substantially the entire reaction period, i.e. at least until all of the brominating agent and compound of Formula (IV-a) have been mixed together. The temperature control is preferably maintained by portionwise addition of brominating agent to the compound of Formula (IV-a) due to bromination reaction is exothermic.

In an embodiment, the present invention provides the compound of formula (III), (IV) ad (V) wherein X is halogen and R⁷ is hydrogen, C₁-C₄ alkyl, which could be prepared and isolated as described above:

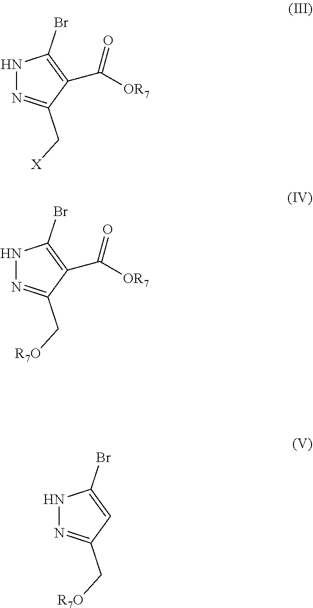

A compound of formula (VII) as well as different methods of its preparation are previously disclosed for example in WO 2003/015519, WO2003016283 and WO 2003/015518.

According to the embodiment of the present invention, the compound of formula (VII), wherein R⁵, R⁶ are as defined above is prepared by reaction of compound of formula (I) with an oxidant.

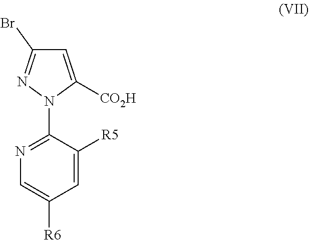

The aforementioned oxidation reaction comprises a solvent selected from water, inert C₁-C₆ alcohols, carboxylic acids and esters thereof, chlorinated hydrocarbons, sulfoxides, sulfones, amides, ethers, ketones, pyridine, nitriles and mixtures thereof. When selecting the solvent, partial or complete dissolution of the starting compound of formula (I) is required. The oxidant can be air, oxygen, potassium persulfate, sodium persulfate, ammonium persulfate, potassium monopersulfate (e.g., Oxone®), sodium permanganate, potassium permanganate and the mixtures thereof. Preferably, the oxidant is potassium permanganate. To obtain complete conversion, at least one equivalent of oxidant versus the compound of formula (I) should be used, preferably from about one to two equivalents. This oxidation is typically carried out in the presence of a solvent. The solvent can be selected from water, inert alcohols, carboxylic acids and esters thereof, chlorinated hydrocarbons, sulfoxides, sulfones, amides, ethers, ketones, pyridine, and mixtures thereof. In an embodiment, the oxidation reaction solvent is selected from an ether, such as tetrahydrofuran, dioxane and the like, an organic ester, such as ethyl acetate, dimethyl carbonate and the like, C₁-C₆ alcohols, such as tert-butanol, or a polar aprotic organic solvents such as N,N-dimethylformamide, acetonitrile and the mixtures thereof. Two or more of the above-mentioned solvents may be used as a mixture, and the reaction may be performed in a single-phase system or a two-phase system. The reaction can be carried out by mixing the compound of Formula (I) in the desired solvent and oxidant, which can be added at a convenient rate. The reaction temperature is typically varied from as low as about 20° C. up to 120° C. in order to obtain a reasonable reaction time to complete the reaction.

According to an embodiment the oxidation reaction is employed in the presence of catalyst. The suitable catalyst, is selected from the group consisting of N-hydroxysuccinimide, N-hydroxyphthalimide, N-hydroxybenzotriazole, quaternary ammonium salts such as tetraethylammonium hydrogensulfate, triethylbenzylammonium chloride, phosphonium salts, such as tetraphenylphosphonium bromide, PEGs, crown ethers, sodium nitrite, tert-butyl nitrite, cobalt(II) acetate, manganese(II) acetate and mixtures thereof.

According to the present invention, the compound of Formula I preferably contacted with the oxidant at raised temperature, i.e. over room temperature (20° C.). A preferred temperature interval is from 40° C. to 120° C., the most preferred interval is from 50° C. to 110° C. Without limiting the scope of protection, the raised temperature most likely promotes the dissolution of the compound of Formula I for more effective oxidation.

The desired product, a compound of formula (VII), can be isolated by methods known to those skilled in the art, including crystallization, extraction and distillation.

In another aspect of this invention, a compounds of Formula (I), (III), (IV), (V), (VII) prepared by the methods of the present invention can be useful as intermediates for preparing the compounds of Formula (VIII)

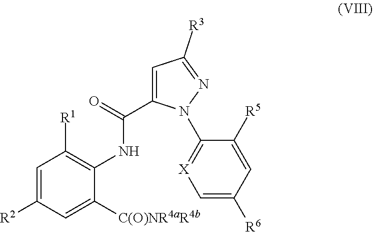

wherein

X is N; R¹ is CH₃, Cl, Br or F;

R² is H, F, Cl, Br or CN;

R³ is Br;

R^{4a} is H, C₁-C₄ alkyl, cyclopropylmethyl or 1-cyclopropylethyl;

R^{4b} is H or CH₃; R⁵ is H, F, CI or Br; and

R⁶ is H, F, CI or Br, by methods known for example from WO 2001/070671, WO 2006062978, WO 2003/015519 and WO 2003/015518.

The following examples are presented in order to illustrate certain embodiments of the invention. They should in no way, however, be construed as limiting the broad scope of the invention. One skilled in the art can readily devise many variations and modifications of the principles disclosed herein without departing from the spirit and scope of the invention.

### EXPERIMENTAL PART

### Examples

### Example 1: Preparation of ethyl (E)-4-chloro-2-((dimethylamino)methylene)-3-oxobutanoate

41.8 g (0.286 mol) 98% ethyl (Z)-3-(dimethylamino)acrylate, and 29 g picoline (0.315 mol) in 50 mL toluene, were mixed and cooled to 0° C. Then 36 g 2-chloroacetyl chloride (0.315 mol) in 50 mL toluene was added dropwise into the reaction within 1 h at −5-0° C. Then the mixture was kept at 25° C. for additional 3 h. 100 mL water was added to quench the reaction, and the organic phase was extracted with toluene. The combined toluene solution was used without purification.

### Example 2: Preparation of ethyl 3-(chloromethyl)-1H-pyrazole-4-carboxylate

Ethyl (E)-4-chloro-2-((dimethylamino)methylene)-3-oxobutanoate prepared by Example 1 was added dropwise to the mixture of 70 g N₂H₄ (20%, 0.286 mol) in 50 mL toluene, during 2 h, and the reaction temperature was kept at 0° C. The mixture was stirred for additional 1 h after the completion of addition. The crude solid was filtrated and then washed with 30 mL toluene and 30 mL H₂O to afford 37.7 g of ethyl 3-(chloromethyl)-1H-pyrazole-4-carboxylate as yellow solid.

### Example 3: Preparation of ethyl 5-bromo-3-(chloromethyl)-1H-pyrazole-4-carboxylate (III)

13 g (0.069 mol) of ethyl 3-(chloromethyl)-1H-pyrazole-4-carboxylate (II) prepared in Example 2 in 50 mL of acetonitrile were heated to 80° C., and 3.2 g of NBS was added to the reaction and the mixture has been stirred at 80° C. for 12 h. Acetonitrile was removed under reduced pressure and the remained oil was stirred in 20 mL methyl tert-butyl ether/n-heptane (1:2) at 25° C. The filtered cake was dried to afford ethyl 5-bromo-3-(chloromethyl)-1H-pyrazole-4-carboxylate 14.8 g as a pale yellow solid.

### Example 4: Preparation of ethyl 5-bromo-3-(methoxymethyl)-1H-pyrazole-4-carboxylate

14.5 g (0.054 mol) of 5-bromo-3-(chloromethyl)-1H-pyrazole-4-carboxylate prepared by Example 3, in 60 mL of methanol and 5.3 g NaHCO₃ (1.168 mol) in 10 mL H₂O were mixed at 10° C. and the resulting mixture was stirred for 4 h. Then methanol was removed under reduced pressure, and the crude product was filtrated and dried to afford 10.7 g of ethyl 5-bromo-3-(methoxymethyl)-1H-pyrazole-4-carboxylate as white solid.

### Example 5: Preparation of ethyl 3-(methoxymethyl)-1H-pyrazole-4-carboxylate

18 g (0.095 mol) of ethyl 3-(chloromethyl)-1H-pyrazole-4-carboxylate prepared by Example 2 was dissolved in 20 mL methanol and added dropwise to the mixture of 14 g sodium bicarbonate in 20 mL methanol and 2 mL water at 25° C. The resulted mixture was kept at 25° C. for 3 and then methanol was removed under reduced pressure. The crude product was filtered and dried to afford 13.1 g of ethyl 3-(methoxymethyl)-1H-pyrazole-4-carboxylate as white solid.

### Example 6: Preparation of ethyl 5-bromo-3-(methoxymethyl)-1H-pyrazole-4-carboxylate

10 g (0.054 mol) of ethyl 3-(methoxymethyl)-1H-pyrazole-4-carboxylate prepared by Example 5 in 50 mL of acetonitrile was heated to 80° C., and then 11 g of NBS was added to the reaction mixture and stirred. Then acetonitrile was distilled out under reduced pressure and the remained oil was stirred in methyl tert-butyl ether/n-heptane (1:2) mixture at 25° C. The crude product was filtrated and dried to give 9.9 g of ethyl 5-bromo-3-(methoxymethyl)-1H-pyrazole-4-carboxylate as white solid.

### Example 7: Preparation of 5-bromo-3-(methoxymethyl)-1H-pyrazole

6.3 g (0.024 mol) of 5-bromo-3-(methoxymethyl)-1H-pyrazole-4-carboxylate prepared by Example 4 was mixed with 40 mL of 40% H₂SO₄, and the reaction mixture was stirred at 100° C. for 30 h. Afterwards, the pH of the reaction mixture was adjusted to pH 7 with NaOH aqueous solution and the product was extracted with ethyl acetate, concentrated and purified by silica gel column to afford 2.5 g of 5-bromo-3-(methoxymethyl)-1H-pyrazole as white solid.

### Example 8: Preparation of 2-(3-bromo-5-(methoxymethyl)-1H-pyrazol-1-yl)-3-chloropyridine

1 g (5.24 mmol) of 5-bromo-3-(methoxymethyl)-1H-pyrazole prepared by Example 7, 2 g of 2,3-dichloropyridine and 1.8 g potassium carbonate powder were mixed in 10 mL of N,N-dimethylacetamide. The reaction was heated to 160° C. and stirred for 5 hours. Then the reaction was cooled to ambient temperature, filtered to remove undissolved solid and washed with 5 mL of N,N-dimethylacetamide. The resulting brown solution was distilled under reduced pressure and the crude product was purified by silica gel column to afford 1.3 g of 2-(3-bromo-5-(methoxymethyl)-1H-pyrazol-1-yl)-3-chloropyridine as pale yellow solid.

### Example 9: Preparation of 3-bromo-1-(3-chloropyridin-2-yl)-1H-pyrazole-5-carboxylic acid

0.6 g (2.0 mmol) of 2-(3-bromo-5-(methoxymethyl)-1H-pyrazol-1-yl)-3-chloropyridine prepared in Example 8 and 5 mL of tert-butyl alcohol were mixed and the reaction mixture was heated to 80° C. After that, 0.6 g of potassium permanganate was dissolved in 5 g H₂O at 60° C., and then added dropwise to the reaction mixture and kept at 80° C. for additional 2 h. Afterwards, the mixture was cooled to room temperature and filtered to remove MnO₂. Aqueous layer was extracted with ethyl acetate and then acidified with 35% vol. HCl. The crude product was filtrated and dried to give 0.3 g of 3-bromo-1-(3-chloropyridin-2-yl)-1H-pyrazole-5-carboxylic acid as white solid.

### Example 10: Preparation of 5-bromo-3-(methoxymethyl)-1H-pyrazole-4-carboxylic acid

3.2 g NaOH in 10 mL H₂O and 11 g (42.8 mmol) of ethyl 5-bromo-3-(methoxymethyl)-1H-pyrazole-4-carboxylate were suspended, heated to 100° C. and kept for 2 h. Then the reaction mixture was cooled to 10° C. and quenched by 30% vol. HCl to adjust the pH to 1-2. The obtained mixture was isolated by filtration. The cake was washed with water and dried to give 9.2 g of 5-bromo-3-(methoxymethyl)-1H-pyrazole-4-carboxylic acid as pale-white solid.

### Example 11: Preparation of 5-bromo-3-(methoxymethyl)-1H-pyrazole

Method A:

9.2 g (39.1 mmol) of 5-bromo-3-(methoxymethyl)-1H-pyrazole-4-carboxylic acid in 20 mL 40% H₂SO₄ was kept at 100° C. for 10 h. The reaction mixture was cooled to room temperature and neutralized with 6 mol/L NaOH aqueous solution until pH reached 7 to 8. The mixture was extracted with 40 ml ethyl acetate and the solvent was removed to afford 6.0 g of 5-bromo-3-(methoxymethyl)-1H-pyrazole as pale-yellow oil.

Method B:

9.2 g of 5-bromo-3-(methoxymethyl)-1H-pyrazole-4-carboxylic acid (39.1 mmol) in 20 mL N,N-dimethyl acetamide was kept at 160° C. for 10 h. The reaction mixture was cooled to room temperature and the resulting 5-bromo-3-(methoxymethyl)-1H-pyrazole was isolated from the precipitated crude product as 6.8 g of as pale-yellow oil.

### Example 12: Preparation of 3-bromo-1-(3-chloropyridin-2-yl)-1H-pyrazole-5-carboxylic acid

To a 250 mL four-necked flask equipped with a magnetic stirrer, a thermometer, a condenser and an oxygen inlet was charged 10 g of 2-[3-bromo-5-(methoxymethyl)-1H-pyrazol-1-yl]-3-chloropyridine, Co(OAc)2 (0.59 g, 10 mol %) NaBr (0.07 g, 0.02 eq) and 80 mL of acetic acid. The mixture was heated to 120° C., while oxygen was bubbled. The reaction was kept at 120° C. for 2 h. After the reaction was finished, it was cooled to room temperature and concentrated to dry. The residue was dissolved in 2 mol/L NaOH aqueous solution, washed with ethyl acetate 30 mL. The aqueous solution was adjusted pH to 1-2 with 32% HCl. The obtained mixture was isolated by filtration and the filtered cake was washed with 20 mL water and dried to give 3-bromo-1-(3-chloropyridin-2-yl)-1H-pyrazole-5-carboxylic acid 8.5 g as an off-white solid (85% yield).

### Example 13: Preparation of 3-bromo-1-(3-chloropyridin-2-yl)-1H-pyrazole-5-carboxylic acid

To a 250 mL four-necked flask equipped with a magnetic stirrer, a thermometer, a condenser and an oxygen inlet was charged 2-[3-bromo-5-(methoxymethyl)-1H-pyrazol-1-yl]-3-chloropyridine 10 g, N-Hydroxysuccinimide (0.22 g, 0.04 eq) and acetic acid 80 mL. The mixture was heated to 120° C., while oxygen was bubbled into and HNO₃ (2.5 ml) was added dropwise. The reaction was kept at 120° C. for 2 h. After the reaction was finished, it was cooled to room temperature and concentrated to dry mass. The dry residue was dissolved in 2 mol/L NaOH aqueous solution, washed with ethyl acetate 30 mL. The aqueous solution was adjusted pH to 1-2 with 32% HCl. The obtained mixture was isolated by filtration and the filtered cake was washed with 20 mL water and dried to give 3-bromo-1-(3-chloropyridin-2-yl)-1H-pyrazole-5-carboxylic acid 8.1 g as an off-white solid (81% yield).

### Example 14: Preparation of 3-bromo-1-(3-chloropyridin-2-yl)-1H-pyrazole-5-carboxylic acid

To a 10 mL three-necked flask was charged 0.6 g of 2-[3-bromo-5-(methoxymethyl)-1H-pyrazol-1-yl]-3-chloropyridine and 5 mL of tert-butanol and the mixture was heated to 80° C. After that, 0.6 g of KMnO₄ (3 eq) was dissolved in Sg H₂O at 60° C. and added dropwise to the reaction mixture. The reaction was kept stirring for another 2 h, then cooled to room temperature, and filtered through a pad of celite to remove MnO₂. The pH of aqueous phase was adjusted to 1-2 by addition of HCl (35%). Then the product was isolated via filtration to give 3-bromo-1-(3-chloropyridin-2-yl)-1H-pyrazole-5-carboxylic acid 0.54 g (90% yield).

1. A process for production of compound of formula (I), 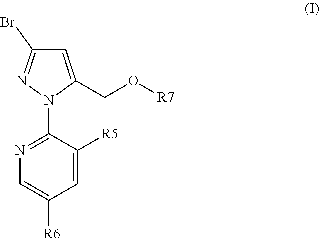
Wherein R⁵ is H, F, Cl or Br; R⁶ is H, F, Cl or Br; R⁷ is hydrogen, C₁-C₄ alkyl, comprising:
a) reaction of compound of formula (II) with brominating agent, optionally in the presence of organic solvent 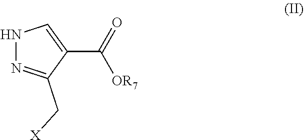
Wherein X is halogen
to prepare a compound of formula (III) 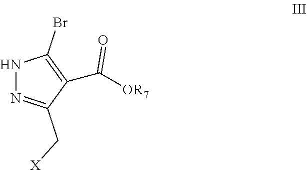
Wherein X is halogen
b) reaction of compound of formula (III) with alkoxylating agent to prepare a compound of formula (IV) 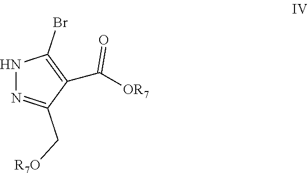
Or, alternatively, a) reaction of compound of formula (II) with alkoxylating agent 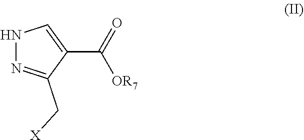
Wherein X is halogen, to prepare a compound of formula (IV-a) 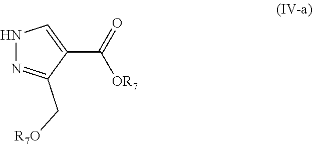
b) bromination of compound (IV-a) to prepare a compound of formula (IV);
c) decarboxylation of compound of formula (IV) to prepare a compound of formula (V): 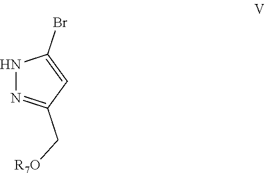
d) reaction of pyridine of formula (VI) 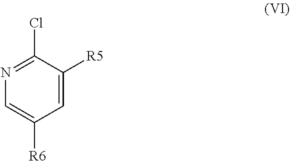
Wherein R⁵ is H, F, Cl or Br; and
R⁶ is H, F, Cl or Br; with compound of formula (V) in the presence of base.

2. The process according to claim 1 wherein the brominating agent is selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric Acid (=DBI), bromodimethylsulfonium bromide, 5,5-dibromomeldrum's acid CAS RN: 66131-14-4, bis(2,4,6-trimethylpyridine)-bromonium hexafluorophosphate, bromine monochloride and the mixtures thereof.

3. The process according to claim 1 wherein the brominating agent is NBS or Br₂.

4. The process according to claim 1 wherein the alkoxylating agent is selected from the group consisting of alkali metal alkoxides of C₁-C₄ alcohols, or C₁-C₄ alcohols in the presence of a base.

5. The process according to claim 1 wherein the base is selected from the group consisting of sodium methoxide, potassium methoxide, sodium ethoxide, potassium ethoxide, potassium tert-butoxide, lithium tert-butoxide, potassium carbonate, sodium bicarbonate, potassium bicarbonate, sodium carbonate, lithium carbonate, sodium hydroxide, lithium hydroxide, potassium hydroxide and the mixtures thereof.

6. The process according to claim 1 wherein the organic solvent is selected from the group consisting of optionally halogenated aromatic hydrocarbons, optionally halogenated hydrocarbons, ketones, nitriles, esters, amides, C₁-C₆ alcohols, sulfones, sulfoxides, carbonates, urea and the mixtures thereof.

7. A process for preparation of compound (VII) 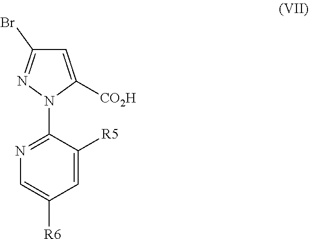
Wherein R⁵ is H, F, Cl or Br; and
R⁶ is H, F, Cl or Br;
comprising reaction of the compound of formula (I) with an oxidant.

8. The process according to claim 7 wherein the oxidant is selected from the group consisting of oxygen, air, ozone, hydrogen peroxide, benzoyl peroxide, tert-butyl peroxide, m-chloroperoxybenzoic acid, peroxyacetic acid, peroxybenzoic acid, magnesium monoperoxyphthalate, potassium peroxymonosulfate, sodium permanganate, potassium permanganate and the mixtures thereof.

9. The process according to claim 7 wherein the compound of formula (VII) is obtained by reacting of compound of formula (I) with an oxidant in the present of catalyst.

10. The process according to claim 9 wherein the catalyst is selected from the group consisting of N-hydroxysuccinimide, N-hydroxyphthalimide, N-hydroxybenzotriazole, tetraethylammonium hydrogensulfate, triethylbenzylammonium chloride, tetraphenylphosphonium bromide, PEGs, crown ethers, sodium nitrite, tert-butyl nitrite, cobalt(II) acetate, manganese(II) acetate and mixtures thereof.

11. The process according to claim 7 performed in the presence of solvent selected from the group consisting of water, C₁-C₆ alcohol, carboxylic acids and esters thereof, chlorinated hydrocarbons, sulfoxides, sulfones, amides, ethers, ketones, nitriles, pyridine, and mixtures thereof.

12. The process according to claim 11 wherein the solvent is selected from the group consisting of water, tert-butanol, tetrahydrofuran, ethyl acetate, N,N-dimethylformamide, acetonitrile and the mixtures thereof.

13. A compound of formula (III): 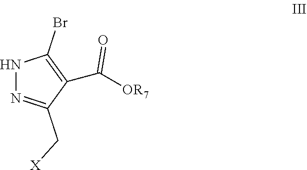
Wherein X is halogen, R₇ is hydrogen, C₁-C₄ alkyl, or
a compound of formula (IV): 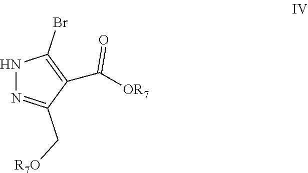
wherein R⁷ is hydrogen, C₁-C₄ alkyl, or
a compound of formula (V): 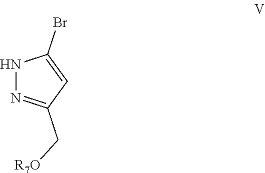
wherein R⁷ is hydrogen, C₁-C₄ alkyl.

14. (canceled)

15. (canceled)

16. A method for preparing of anthranilamide of formula (VIII), 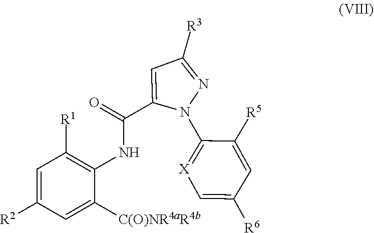
wherein
X is N; R¹ is CH₃, Cl, Br or F;
R² is H, F, Cl, Br or CN;
R³ is Br;
R^{4a} is H, C₁-C₄ alkyl, cyclopropylmethyl or 1-cyclopropylethyl;
R^{4b} is H or CH₃; R⁵ is H, F, Cl or Br; and
R⁶ is H, F, Cl or Br,
using compound of formula (I) prepared according to claim 1.

17. A method for preparing of anthranilamide of formula (VIII), 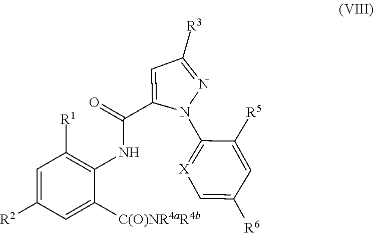
wherein
X is N; R¹ is CH₃, Cl, Br or F;
R² is H, F, Cl, Br or CN;
R³ is Br;
R^{4a} is H, C₁-C₄ alkyl, cyclopropylmethyl or 1-cyclopropylethyl;
R^{4b} is H or CH₃; R⁵ is H, F, Cl or Br; and
R6 is H, F, Cl or Br,
using compound of formula (VII) prepared according to claim 7.

18. A method for preparing of anthranilamide of formula (VIII) 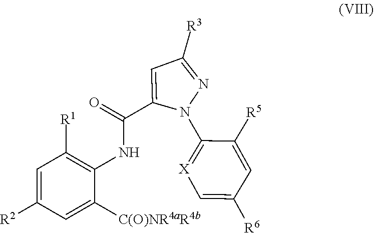
wherein
X is N; R¹ is CH₃, Cl, Br or F;
R² is H, F, Cl, Br or CN;
R³ is Br;
R^{4a} is H, C₁-C₄ alkyl, cyclopropylmethyl or 1-cyclopropylethyl;
R^{4b} is H or CH₃; R⁵ is H, F, Cl or Br; and
R⁶ is H, F, Cl or Br,
using compound of formula (III) prepared according to claim 1.

19. A method for preparing of anthranilamide of formula (VIII) 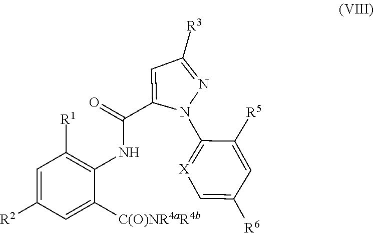
wherein
X is N; R¹ is CH₃, Cl, Br or F;
R² is H, F, Cl, Br or CN;
R³ is Br;
R^{4a} is H, C₁-C₄ alkyl, cyclopropylmethyl or 1-cyclopropylethyl;
R^{4b} is H or CH₃; R⁵ is H, F, Cl or Br; and
R⁶ is H, F, Cl or Br,
using compound of formula (IV) prepared according to claim 1.

20. A method for preparing of anthranilamide of formula (VIII) 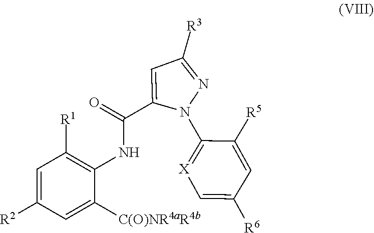
wherein
X is N; R¹ is CH₃, Cl, Br or F;
R² is H, F, Cl, Br or CN;
R³ is Br;
R^{4a} is H, C₁-C₄ alkyl, cyclopropylmethyl or 1-cyclopropylethyl;
R^{4b} is H or CH₃; R⁵ is H, F, Cl or Br; and
R⁶ is H, F, Cl or Br,
using compound of formula (V) prepared according to claim 1.
